(19) United States
(12) Patent Application Publication
(10) Pub. No.: US 2025/0319022 A1
(43) Pub. Date: Oct. 16, 2025

(54) CONCENTRATED SOLUTIONS COMPRISING SODIUM 5,10-METHYLENE-(6R)-TETRAHYDROFOLATE

(71) Applicant: Merck Patent GMBH, Darmstadt (DE)

(72) Inventors: Rudolf Moser, Schaffhausen (CH); Viola Groehn, Dachsen (CH); Thomas Ammann, Marthalen (CH); Jean-Pierre Knapp, Schaffhausen (CH); Marianne Svaerd, Veberoed (SE)

(21) Appl. No.: 18/869,139

(22) PCT Filed: Jun. 5, 2023
(86) PCT No.: PCT/EP2023/064970

§ 371 Date: Nov. 25, 2024

(30) Foreign Application Priority Data
Jun. 8, 2022 (EP) 22177930.9

(51) Int. Cl. A61K 9/08; A61K 9/00; A61K 31/519; A61K 47/02

(52) U.S. Cl. CPC A61K 9/08; A61K 9/0019; A61K 31/519; A61K 47/02

(57) ABSTRACT

The present invention relates to liquid compositions comprising a high content of the sodium salt of 5,10-methyl-ene-(6R)-tetrahydrofolic acid and sulfate, which formulations and lyophilizates do not contain any extraneous stabilizers.

The present invention relates to liquid compositions comprising a high content of the sodium salt of 5,10-methylene-(6R)-tetrahydrofolic acid (5,10-CH₂-(6R)-THF*Na) and sulfate, which formulations and lyophilizates do not contain any extraneous stabilizers.

### BACKGROUND OF THE INVENTION

5,10-methylenetetrahydrofolic acid is known as a medicament used in combination with 5-fluorouracil (5-FU) in the treatment of solid tumors (Seley, K. L. Drugs 4 (1), 99, 2001). The active isomeric form 5,10-methylene-(6R)-tetrahydrofolic acid (referred to as 5,10-CH₂-(6R)-THF in the following), achieves its chemotherapeutic effect together with the base analogue and 5-FU metabolite 5-FdUMP by inhibiting the enzyme thymidylate synthase (TS). TS catalyses the conversion of deoxyuridylate (dUMP) to deoxythymidylate (dTMP), which is an essential building block for DNA synthesis. Deactivation of TS occurs by formation of a covalent, ternary inhibition complex between TS, the base analogue 5-FdUMP, and 5,10-CH₂-(6R)-THF.

An enhancement of the cytotoxic effect of 5-FU can be achieved by increasing the intracellular concentration of 5,10-CH₂-(6R)-THF, whereupon the stability of the ternary inhibition complex is increased. This causes direct inhibition of DNA synthesis and repair, which ultimately results in cell death and delay of tumor growth. In order to achieve high intracellular concentrations of 5,10-CH₂-(6R)-THF the application of respective stable, high content products is desired.

However, there are undesirable properties associated with 5,10-CH₂-(6R)-THF that limit its pharmaceutical use. For example, 5,10-CH₂-(6R)-THF is highly susceptible to oxidation and chemical degradation that results in insufficient stability and unfavourably high levels of impurities.

5,10-methylenetetrahydrofolic acid is an addition product of tetrahydrofolic acid and formaldehyde (see e.g. Poe, M. et al. Biochemistry 18 (24), 5527, 1979; Kallen, R. G. Methods in Enzymology 18B, 705, 1971) and is known for its extremely high sensitivity to oxidation by air as well as instability in neutral and/or acidic environments potentially leading to chemical degradation and/or hydrolysis (see e.g. Odin, E. et al., Cancer Investigation 16 (7), 447, 1998;

Osborn, M. J. et al., J. Am. Chem. Soc. 82, 4921, 1960; Hawkes, J., and Villota, R. Food Sci. Nutr. 28, 439, 1989). Susceptibility to oxidation, chemical degradation and insufficient stability of 5,10-CH₂-(6R)-THF is especially apparent in aqueous solution, or when the compound is present in its amorphous form where it has a large surface (e.g. in its pharmaceutical use form as a lyophilizate), or in re-dissolved form such as solutions for injection. It is well known that to be amenable for pharmaceutical use, the respective composition needs to fulfill several requirements including high chemical and isomeric stability, such that effective storage over an acceptable period of time can be achieved, without exhibiting a significant change in the composition's physicochemical characteristics, ease of handling and processing, etc.

Attempts to stabilize compositions of 5,10-methylenetetrahydrofolates included e.g. (i) rigorous exclusion of atmospheric oxygen by the use of special technical devices for the reconstitution of solid formulations and the injection of 5,10-methylenetetrahydrofolates in an air-free environment (see e.g. Odin, E. et al., Cancer Investigation 16 (7), 447, 1998; U.S. Pat. No. 4,564,054); (ii) addition of a reducing agent such as L (+)-ascorbic acid or salts thereof, reduced gamma-glutathione, beta-mercaptoethanol, thioglycerol, N-acetyl-L-cysteine, etc. as an antioxidant for the highly sensitive 5,10-methylenetetrahydrofolic acid and for tetrahydrofolic acid in particular; (iii) stabilization by means of cyclodextrin inclusion compounds (see e.g. EP 0 579 996); (iv) addition of citrate while adjusting the pH to a basic value (see e.g. EP 1 641 460); or (v) formation of various crystalline forms such as the sulfate salts (see e.g. EP 0 537 492) or hemisulfate salts (see e.g. EP 2 837 631).

Lyophilizates of 5,10-CH₂-(6R)-THF have as described hereinabove previously been prepared from aqueous solutions which contain—in addition to the active compound, i.e. 5,10-CH₂-(6R)-THF—also dicarboxylic acids and/or tricarboxylic acids such as citric acid and/or other stabilizers, see e.g. WO2019034673, US 2007/0099866 and U.S. Pat. No. 10,059,710 B2. Solutions disclosed therein for the purpose of preparing lyophilizates contain at most 2-3% by weight 5,10-CH₂-(6R)-THF.

However, neither lyophilizates containing dicarboxylic acids and/or tricarboxylic acids such as citric acid and/or other stabilizers, nor the crystalline salt forms of 5,10-methylenetetrahydrofolic acid are readily useful for pharmaceutical purposes due to their low aqueous solubility, and moreover the stabilized versions of 5,10-methylenetetrahydrofolic acid known in the art usually contain less than 50% of the active drug compound 5,10-CH₂-(6R)-THF due the dilution in the final dosage form by the stabilizing additives.

As an example, the company Adventrx Pharmaceuticals carried out stability studies on their drug candidate CoFactor®, i.e. the calcium salt of the diastereomer mixture 5,10-methylene-(6R,S)-tetrahydrofolic acid, which were disclosed i.a. in WO 2007/064968. The chemical stability of the diastereomer mixture 5,10-methylene-(6R,S)-tetrahydrofolic acid is assumed to be similar to the pure diastereomer 5,10-CH₂-(6R)-THF of the present invention. The study compared the stability of nonformulated 5,10-methylene-(6R,S)-tetrahydrofolic acid with 5,10-methylene-(6R,S)-tetrahydrofolic acid formulated with only trisodium citrate or formulated with both ascorbic acid and trisodium citrate; both of which compounds are well-known reducing agents (see FIG. 1).

Linear regression analysis of the stability profiles of the isolated lyophilizates showed that the degradation of 5,10-methylene-(6R,S)-tetrahydrofolic acid was linear over time (see FIG. 2). The degradation rate (slope of the best-fit line) for each formulation (reconstituted lyophilizate) demonstrated the following order, from fastest to slowest degradation rate: nonformulated>formulated with only trisodium citrate>formulated with both ascorbic acid and trisodium citrate (FIG. 2). Nonformulated 5,10-methylene-(6R,S)-tetrahydrofolic acid was thus found to lose 2.3% purity per hour, resulting in a purity of 84% after 7 hours, whereas formulations containing trisodium citrate+ascorbic acid had much higher stability, resulting in a purity of about 95% after 7 hours.

Moreover, the solutions disclosed in WO 2007/064968 for the purpose of preparing the most stable lyophilizates contain less than 5% by weight 5,10-methylene-(6R,S)-tetrahydrofolic acid, and the resulting lyophilizates contain less than 20% by weight 5,10-methylene-(6R,S)-tetrahydrofolic acid (see FIG. 3).

Additionally, stabilizers such as citric acid, used to prepare the most stable lyophilizates in WO 2007/064968, for example, have been linked to various undesired effects like e.g. QTc elongation (Laspina et al. Transfusion 42 (2002) p. 899, Toyoshima et al. Clinical Nutrition (2006) 25, 653-660), inducing hypocalcaemia (Payne et. Al. J. Physiol. (1964), 170, pp. 613-620), etc. From a clinical perspective the availability of stable solutions and lyophilizates of 5,10-CH₂-(6R)-THF having a high content of the active ingredient and being free of any kind of stabilizers would therefore be an advantage.

There thus still remains a great need for stable pharmaceutical compositions having a high content of 5,10-methylene-(6R)-tetrahydrofolic acid.

### SUMMARY OF THE INVENTION

It has now surprisingly been found that aqueous solutions comprising a high content of the sodium salt of 5,10-methylene-(6R)-tetrahydrofolic acid (denoted hereinafter 5,10-CH₂-(6R)-THF*Na) in combination with from more than 40 mol-%, preferably from about 40 mol-% to 200 mol-%, even more preferred from about 50 mol-% to about 100 mol-% of an inorganic, aqueously soluble sulfate salt such as an alkali metal sulfate or alkali metal hydrogensulfate, (in the following referred to just as “sulfate” or “alkali metal sulfate”) can be prepared.

The obtained solutions contain more than 60 mg 5,10-CH₂-(6R)-THF*Na per ml, such as more than 65 mg/ml, more than 70 mg/ml, more than 75 mg/ml, such as preferably at least 80 mg 5,10-CH₂-(6R)-THF*Na per ml. Solutions of higher concentration can be prepared but become very viscous. In the obtained solutions 5,10-CH₂-(6R)-THF*Na represents more than about 60% w/w (or more than 15 mol-%) of the solid material, preferably more than 80% w/w (or more than 30 mol-%).

The solutions of the invention can further be converted in high yields to stable lyophilizates comprising 5,10-CH₂-(6R)-THF*Na and sulfate, which lyophilizates do not contain any stabilizing agents. These lyophilizates are found to have surprisingly high stability, and thus overcome the previously discussed known drawbacks and allow for the preparation of stable solid-state pharmaceutical compositions of high purity and a low content of either oxidation products or other chemical degradation products.

The advantageous stability and concentration characteristics of the concentrated aqueous solutions of the present invention will allow the effective, and safe use in medicinal applications.

In a first aspect the present invention relates to a concentrated aqueous solution which comprises the sodium salt of 5,10-CH₂-(6R)-tetrahydrofolic acid (5,10-CH₂-(6R)-THF*Na) and an alkali metal sulfate, which concentrated aqueous solution further does not contain any stabilizing agents such as buffers, reducing agents and the like, as defined herein.

A second aspect of the present invention is directed to a process for the preparation of a concentrated aqueous solution according to the first aspect, which process comprises the following steps:
i. dissolving (6S)-tetrahydrofolic acid in aqueous NaOH,
ii. adjusting the pH of the solution to 8.6±0.5,
iii. adding 100-120 mol % formaldehyde,
iv. stirring the reaction mixture until reaction has completed,
v. adding a solution of an alkali metal sulfate, and
vi. filtering the reaction mixture to obtain a clear solution of 5,10-CH₂-(6R)-THF*Na and alkali metal sulfate.

In a third aspect the present invention further relates to a concentrated aqueous solution according to the first aspect, or a reconstituted or diluted aqueous solution thereof, for use in the treatment of cancer, or in cancer therapy, in a human patient.

In a fourth aspect the present invention further relates to a method of treatment of cancer, or of cancer therapy, in human patients comprising administering a concentrated aqueous solution according to the first aspect, or a reconstituted or diluted aqueous solution thereof, to a human patient in need thereof.

In a fifth aspect the present invention further relates to the use of a concentrated aqueous solution according to the first aspect, or a reconstituted or diluted aqueous solution thereof, for the manufacture of a medicament for the treatment of cancer in human patients.

The concentrated aqueous solution of the first aspect, comprising the sodium salt of 5,10-methylene-(6R)-tetrahydrofolic acid and sulfate, have a high purity and remains chemically stable for at least 7 hours at 5±3° C. or for at least 3 hours at room temperature, even without sparging the solution with nitrogen for minimizing degradation by oxidation. See FIG. 4. A highly concentrated solution of 75 mg/ml is clear and remains clear regardless if it is stored at 2-8° C. or at RT, i.e., no precipitation occurs.

### BRIEF DESCRIPTION OF THE FIGURES

FIG. 1 is adapted from Table 2 in WO 2007/064968 and demonstrates the stability over time of non-formulated and various formulated forms of 5,10-methylene-(6R,S)-tetrahydrofolic acid (% normalized purity). As can be seen, each formulation had a different stability profile. Thus, non-formulated 5,10-methylene-(6R,S)-tetrahydrofolic acid at neutral pH degraded rapidly over time. 24 hours following dissolution in water, the purity of non-formulated 5,10-methylene-(6R,S)-tetrahydrofolic acid was only 44.9% of the starting purity. The reference formulation formulated only with trisodium citrate (pH adjusted >7.5) showed slower degradation following dissolution in water.

However, purity after 24 hours was still only 65% compared to the starting purity, indicating degradation was not efficiently inhibited by the addition of trisodium citrate and adjustment of pH. The two test formulations #1 and #2 (i.e. 5,10-methylene-(6R,S)-tetrahydrofolic acid formulated with both ascorbic acid and trisodium citrate) were the most stable formulations (purity after 24 hours about 89%).

FIG. 2 is adapted from FIG. 1 in WO 2007/064968 and demonstrates graphically the tabulated results of FIG. 1 herein.

FIG. 3 is a table adapted from Example 1 of WO 2007/064968 showing the composition of the non-formulated and formulated forms of 5,10-methylene-(6R,S)-tetrahydrofolic acid shown in FIG. 1 and FIG. 2 herein.

FIG. 4 shows the purity analyses of four identical solutions of sodium salt of 5,10-methylene-(6R)-tetrahydrofolic acid of the present invention tested at four different conditions: 5° C. without a blanket of N₂, 5° C. with a blanket of N₂, 4 hrs at 5° C. followed by 3 hrs at room temperature with a blanket of N₂, and 4 hrs at 5° C. followed by 3 hrs at room temperature without a blanket of N₂. The results are shown for a total period of 7 hours. As can be seen from the graphs, the solutions are very stable under the storage conditions, changing from an initial purity between 96.6-97% to a purity of 96.4-96.5% (area %). As can also be seen, the effect of N₂ blanketing is minimal.

FIG. 5 shows analyses of the same four solutions of sodium salt of 5,10-methylene-(6R)-tetrahydrofolic acid as shown in FIG. 4 herein. In FIG. 5, the development over 7 hours of the main impurity, 10-formyl-(6R)-tetrahydrofolic acid (10-FTHFA) in the solutions as produced in Example 3 when stored at 2-8° C., is shown. As can be seen, the level of this impurity is practically constant over time.

### DEFINITIONS

As used herein, the term “sulfate” shall refer to an inorganic, aqueously soluble sulfate salt such as an alkali metal sulfate or alkali metal hydrogensulfate.

In the present text, the term “buffer” relates to citrate (or citric acid and salts thereof); dicarboxylates such as succinate, malate and maleate; tris(hydroxymethyl)aminomethane (TRIS); N-tris(hydroxymethyl)methyl-2-aminoethanesulfonic acid (TES); 3-(N-morpholino) propanesulfonic acid (MOPS); N,N-bis(2-hydroxyethyl)-2-aminoethane-sulfonic acid (BES); MES; MOPSO; HEPES; phosphate; carbonate; ammonium; mono-, di- and tri-alkylammonium; mono-, di- and tri-hydroxylalkylammonium; glutamate; borate; lactate; as well as combinations of these.

In the present text, the term “reducing agent” relates to L-(+) ascorbic acid or salts thereof, reduced γ-glutathione, β-mercaptoethanol, thioglycerol, and N-acetyl-L-cysteine.

In the present text, the term “solvent” relates to solvents which may be used in freeze drying processes. “Solutions” as referred to in the present text, comprise aqueous solutions as well as solutions in organic solvents. Typically, “aqueous solutions” mean solutions in water, saline solutions, water containing small amounts of buffers, water containing isotonic amounts of NaCl, or mixtures of water with organic solvents, and the like. Typical organic solvents include DMSO, acetonitrile, acetone, methanol, or ethanol.

### DETAILED DESCRIPTION OF THE INVENTION

It has surprisingly been found that high-content, aqueous solutions comprising the sodium salt of 5,10-methylene-(6R)-tetrahydrofolic acid (denoted 5,10-CH₂-(6R)-THF*Na) and about 40-200 mol-% of an alkali metal sulfate are remarkably stable.

The solutions of the present invention contain more than 60 mg 5,10-CH₂-(6R)-THF*Na per ml, such as more than 65 mg/ml, more than 70 mg/ml, more than 75 mg/ml, such as preferably at least 80 mg 5,10-CH₂-(6R)-THF*Na per ml. Solutions of higher concentration can be prepared but become very viscous.

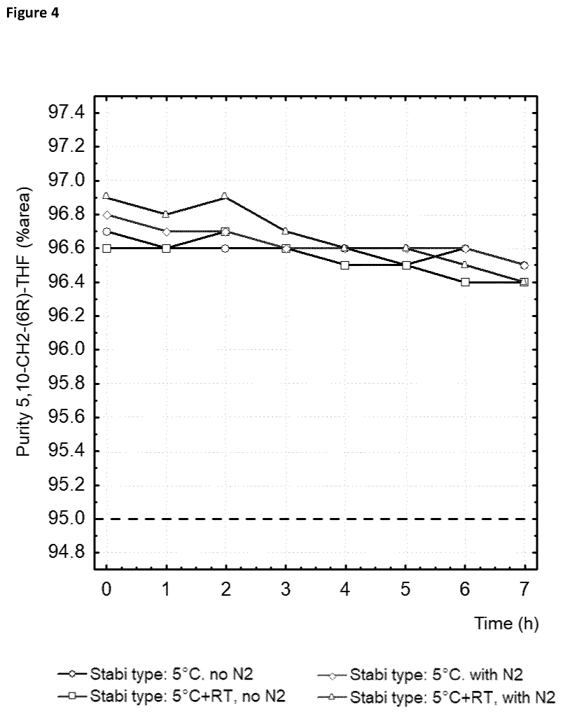

The highly concentrated solutions according to the instant invention are aqueous compositions comprising 5,10-CH₂-(6R)-THF*Na and alkali metal sulfate, as disclosed above. These compositions have a high purity and remain chemically stable for at least 7 hours at 5±3° C. or for at least 3 hours at room temperature, even without sparging the solution with nitrogen for minimizing degradation by oxidation (see FIG. 4). The highly concentrated solution of 75 mg/ml is clear and remains clear regardless if it is stored at 2-8° C. or at RT, i.e. no precipitation occurs.

The aqueous solutions according to the instant invention can be filled in containers and freeze-dried (lyophilized) to a stable, non-sticky lyophilizate powder and stored. The lyophilizate can be reconstituted with a diluent to a set concentration for administration. Alternatively, aqueous solutions can be produced in a “ready to use” concentration and filled in containers, e.g. vials or ampoules. Such solutions, or reconstituted lyophilizates, can be administered either intramuscularly or intravenously.

The solutions of the invention may contain additional excipients. Bulking agents such as mannitol may be added to the solution before the freeze-drying process to promote an acceptable lyophilized cake formation.

Also, electrolytes, sugars and/or polyols such as dextrose, glycerol, mannitol and sodium chloride may be added to the aqueous solutions of the invention to adjust the osmolality.

The pH of the solutions is typically in the range of 8.0 to 9.0, preferably in the range of 8.4 to 8.8, and can be adjusted during drug product manufacturing with e.g. small amounts of hydro-chloric acid or sodium hydroxide.

For longer-term storage it is advantageous to lyophilize the highly concentrated solutions of the instant invention.

Stability is a critical property and component of pharmaceutical formulation studies and drug development. Stability studies are performed both in solution and solid state. It is an established fact that the solution state and solid-state stability can differ both qualitatively and quantitatively. Extensive studies were performed for stability of the drug substance and pharmaceutical compositions thereof by exposing it to variety of stressors, like high temperature and/or high humidity. These studies also provide information on the degradation products and help in developing meaningful specifications as well as the intrinsic stability of the pharmaceutical composition. Most common pathways for drug degradation include i.a. hydrolysis, oxidation, and photochemical degradation.

The purpose of stability testing is to provide evidence on how the quality of a product varies with time under the influence of a variety of environmental factors such as temperature, humidity, and light, and to establish a suitable shelf life for the pharmaceutical product and recommended storage conditions, in order to ensure patient safety.

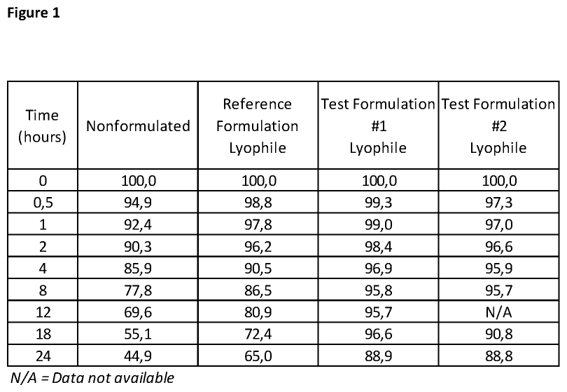

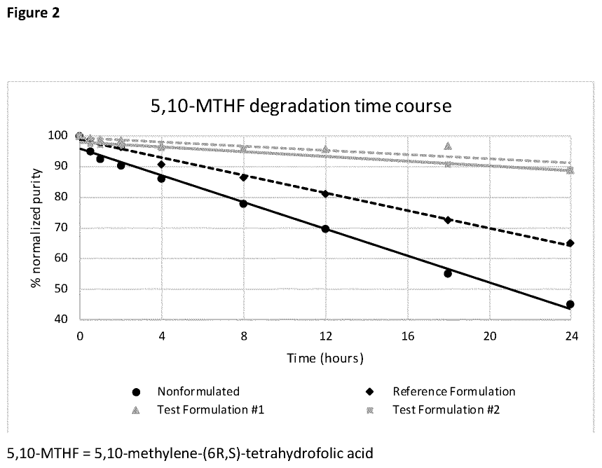

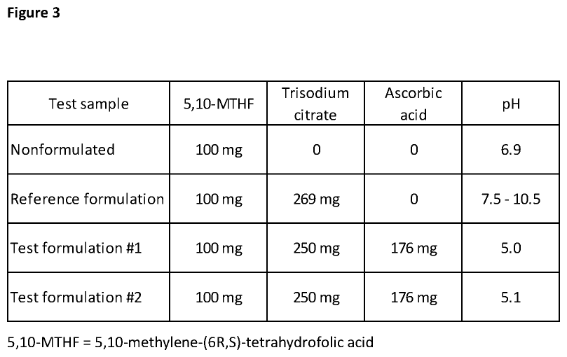

The high stability observed for the concentrated solutions of 5,10-CH₂-(6R)-THF*Na in combination with alkali metal sulfate is highly surprising in view of the art described above, in which the presence of a stabilizer like citrate would have been mandatory. A comparison of FIG. 4 with FIGS. 1-3 thus strongly indicates that the high-content solutions of the present invention have similar or better stability than the ascorbate/citrate stabilized CoFactor® compositions discussed in i.a. WO 2007/064968.

In a first aspect the present invention relates to a concentrated aqueous solution which comprises the sodium salt of 5,10-CH₂-(6R)-tetrahydrofolic acid (5,10-CH₂-(6R)-THF*Na) and an alkali metal sulfate, which concentrated aqueous solution further does not contain any stabilizing agents such as buffers, reducing agents and the like, as defined herein.

The concentrated aqueous solution of the present invention are preferably reconstituted by dilution into an aqueous pharmaceutical formulation to be administered into a patient in need thereof.

The present invention in one embodiment discloses a concentrated aqueous solution according to the first aspect wherein the molar ratio of alkali metal sulfate:5,10-CH₂-(6R)-THF is from about 0.4:1 to about 1:2, preferably from about 0.5:1 to about 1:1.

A second aspect of the present invention is directed to a process for the preparation of an aqueous solution comprising the sodium salt of 5,10-CH₂-(6R)-tetrahydrofolic acid and an alkali metal sulfate, which process comprises the following steps:
i. dissolving (6S)-tetrahydrofolic acid in water at about pH 11,
ii. adjusting the pH of the clear solution to 8.6±0.5,
iii. adding 100-120 mol % formaldehyde,
iv. stirring the reaction mixture until reaction has completed,
v. adding an alkali metal sulfate,
vi. filtering the reaction mixture to obtain a clear solution of 5,10-CH₂-(6R)-THF*Na and alkali metal sulfate.

The reaction between (6S)-tetrahydrofolic acid and formaldehyde is quantitative, but it is advisable to employ a slight excess of formaldehyde to ensure that the reaction goes to completion. It should be avoided to employ too much formaldehyde, as this leads to increased levels of impurities (cf. Example 2a and 2b herein).

In a preferred embodiment of the second aspect, an alkali metal sulfate is added in step v. up to a final ratio of alkali metal sulfate:5,10-CH₂-(6R)-tetrahydrofolic acid from about 0.4:1 to about 1:2.

In a preferred embodiment of the second aspect, about 110 mol % formaldehyde is employed.

In another preferred embodiment, the alkali metal sulfate added in step v. is sodium sulfate.

Once the solution of 5,10-CH₂-(6R)-THF*Na has been generated, i.e. from step iv.-v., the temperature of the reaction mixture should be kept low, preferably around 0-5° C.

In a third aspect the present invention further relates to a concentrated aqueous solution according to the first aspect, or a reconstituted or diluted aqueous solution thereof, for use in the treatment of cancer, or in cancer therapy, in a human patient.

In a fourth aspect the present invention further relates to a method of treatment of cancer, or of cancer therapy, in human patients comprising administering a concentrated aqueous solution according to the first aspect, or a reconstituted or diluted aqueous solution thereof, to a human patient in need thereof.

In a preferred embodiment the present invention relates to a method of treatment of cancer in human patients comprising administering a concentrated aqueous solution according to the first aspect, or a diluted aqueous solution thereof, to a human patient in need thereof.

In a sixth aspect the present invention further relates to the use of a composition comprising 5,10-CH₂-(6R)-THF*Na and an alkali metal sulfate according to the first aspect, or reconstituted or diluted aqueous solutions thereof, for the manufacture of a medicament for the treatment of cancer in human patients.

A further aspect is thus directed to reconstituted pharmaceutical compositions of the concentrated aqueous solutions of the present invention comprising 5,10-CH₂-(6R)-THF*Na, an alkali metal sulfate and a pharmaceutically acceptable carrier or diluent, such as sterile water or a liquid pharmaceutically acceptable vehicle, optionally further comprising at least one additional therapeutic agent including but not limited to, bactericides, antibiotics, antivirals, antiseptics, antineoplastics, anticancer compounds such as chemotherapeutic agents, antifungals, and/or anti-inflammatory agents or other bioactive or therapeutic agents that are suitable for human use, in particular anticancer compounds such as chemotherapeutic agents, for example 5-FU and derivatives, and antifolates, e.g. methotrexate, Pemetrexed.

### EXAMPLES

### HPLC

For the measurement of purity/content and degradation products an HPLC-UV Gradient Method was used: Column type: ODS, Mobile phase: A: aqueous Buffer; Mobile Phase: B: aqueous Buffer/Methanol, Run time: 30 min, Sample Solvent: aqueous Buffer.

### Water Content

The determination of the water content was performed according to Ph. Eur. 2.5.32/USP <921/Method Ic>.

### Osmolality

The determination of the osmolality was performed according to Ph. Eur. 2.2.35 (osmometer)/USP <785>.

### Example 1: Preparation of a Concentrated Aqueous Solution Comprising Sulfate and Sodium 5,10-methylene-(6R)-tetrahydrofolate

(a) 7.93 g (16 mmol) (6S)-tetrahydrofolic acid and 78.0 g distilled water were provided in a roundbottom flask at room temperature under N₂. The resulting suspension was stirred, and the pH adjusted to pH 11 by slow addition of a 32% NaOH solution. As soon as the solution became clear, a 1 M HCl solution was added gradually to adjust the pH of the reaction mixture to 8.3 at 25° C. The obtained clear solution was cooled to about 0° C., at which temperature it showed a pH of 8.8. The pH was again adjusted with 1 M HCl to pH=8.6 and 1.44 g of a 36.8% HCHO solution (110 mol %) was added in one portion. Upon completion of the addition the solution was stirred at 0° C. (ice bath) for 1 hour. Active charcoal (0.2 g, Norit C Extra) was added and the reaction mixture was stirred for 30 minutes at 0° C. and then cold filtered over a suction filter to obtain a clear solution of 5,10-CH₂-(6R)-THF*Na, which was used in step (b) without further purification.
(b) A chilled solution of 2.8 gr Na₂SO₄ (20 mmol, 1.25 mol %) in 15 ml distilled water was added to the solution as obtained in step (a). The pH was then adjusted with 1 M NaOH to 9.3±0.1, and the obtained reaction mixture was stirred under N₂ at 0° C. for 2 hours. Active charcoal (0.2 g, Norit C Extra) was added and the reaction mixture was stirred for 30 minutes at 0° C. and then cold filtered over a suction filter followed by sterile filtration through a 0.22 μm filter to obtain a clear solution of an approximately 1:1 molar composition of sodium 5,10-CH₂-(6R)-THF*Na and sodium sulfate. The solution contains about 8 gr 5,10-CH₂-(6R)-THF*Na per 100 ml, i.e. a concentration of about 80 mg/ml, corresponding to about 7.3 gr 5,10-CH₂-(6R)-THF free acid in 100 ml. The solution should be kept at 2-8° C.
(c) Cool the solution from step (b) to 2-8° C. and pass it through a 0.22 μm filter while keeping the solution as cold as possible. Fill the filtered solution into glass vials (2 ml or 160 mg 5,10-CH₂-(6R)-THF*Na per vial) while keeping the solution as cold as possible.

The influence of formaldehyde excess on product quality was analysed in the two following examples which were carried out identically except from the excess of formaldehyde. In example 2a, 110 mol % formaldehyde was used, whereas in example 2b 200 mol % formaldehyde was used. The use of 110 mol % formaldehyde in Example 2a provided the purest product.

### Example 2a: Preparation of a 5,10-methylene-(6R)-tetrahydrofolate with sulfate

4.72 g (6S)-Tetrahydrofolic acid were added under nitrogen to 220 ml water containing 10 g NaOH 2M (initial pH 13.74). The pH was kept at 9.3±0.1 until complete dissolution with 22.8 g NaOH 2M. Then 0.901 g of a 36.8% HCHO solution were added (110 mol %). The solution was stirred for 30 minutes. A chilled solution of 4.5 gr Na₂SO₄ (20 mmol, 1.25 mol %) in 15 ml distilled water was added to the solution and thereafter the pH was adjusted again to 9.3 with aqueous sodium hydroxide 2M (0.05 g). The so obtained solution contained 5,10-methylene-(6R)-tetrahydrofolic acid and sulfate with a purity of 94.8% area.

### Example 2b: Preparation of a 5,10-methylene-(6R)-tetrahydrofolate with sulfate

4.72 g (6S)-Tetrahydrofolic acid were added under nitrogen to 220 ml water containing 10 g NaOH 2M (initial pH 13.83). The pH was kept at 9.3±0.1 until complete dissolution with 22.8 g NaOH 2M. Then 1.639 g formaldehyde solution (36.76%) were added (200 mol %). The solution was stirred for 30 minutes. A chilled solution of 4.5 gr Na₂SO₄ (20 mmol, 1.25 mol %) in 15 ml distilled water was added to the solution and thereafter the pH was adjusted again to 9.3 with aqueous sodium hydroxide 2M. The so obtained solution contained 5,10-methylene-(6R)-tetrahydrofolic acid and sulfate with a purity of 91.5% area.

### Example 3: Preparation of a Stabilizer-Free Lyophilisate

Fill the filtered solution from Example 1 at a temperature of 2-8° C. into vials (2 ml or 150 mg 5,10-CH₂-(6R)-THF per vial) while keeping the solution as cold as possible. Freeze-dry the vials and seal them under a slight vacuum with nitrogen in the headspace. Crimp the vials. The resulting lyophilisate contains 70-80% w/w 5,10-CH₂-(6R)-THF.

### Example 4: Stability Testing

The solutions as produced in Example 1, step c, were tested for stability under four different conditions: 7 hrs at 5° C. without a blanket of N₂, 7 hrs at 5° C. with a blanket of N₂, 4 hrs at 5° C. followed by 3 hrs at room temperature with a blanket of N₂, and 4 hrs at 5° C. followed by 3 hrs at room temperature without a blanket of N₂. The results are shown in FIG. 4. As can be seen from the graphs, the solutions are very stable under the storage conditions, changing from an initial purity between 96.6-97% to a purity of 96.4-96.5% (area %). As can also be seen from FIG. 4, the effect of N₂ blanketing on stability is minimal.

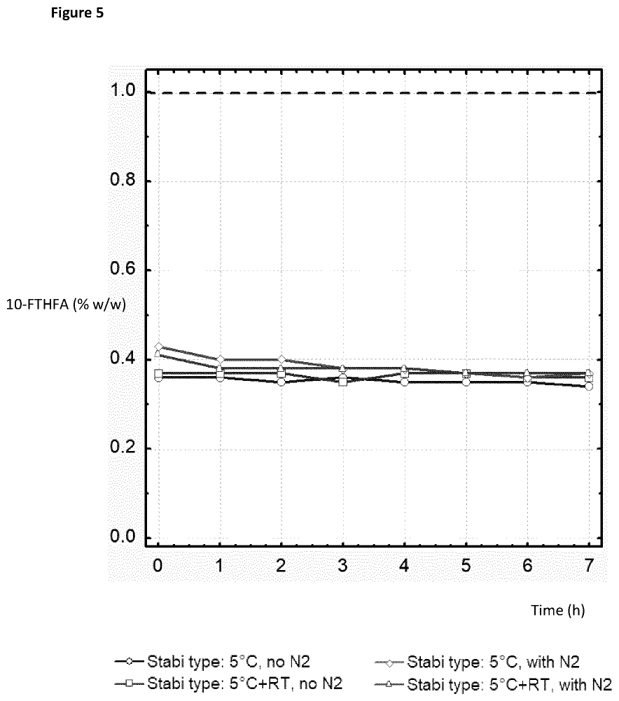

As part of the stability analysis, the development over 7 hours of the main impurity, 10-formyl-(6R)-tetrahydrofolic acid (10-FTHFA) in the solutions as produced in Example 1, step c when stored at 2-8° C. was also measured (see Table 1 below and FIG. 5). As can be seen, the level of this impurity is practically constant.

TABLE 1
Analysis of impurities in a solution comprising sodium
5,10-methylene-(6R)-tetrahydrofolic acid and
sulfate (main degradation product)

| | Content of 10-formyl-(6R)-tetrahydrofolic | | | | | | | |
|---|---|---|---|---|---|---|---|---|
| Time (hrs) | acid over storage time at 2-8° C. | | | | | | | |
| at 2-8° C. | 0 | 1 | 2 | 3 | 4 | 5 | 6 | 7 |
| (% w/w) | 0.36 | 0.36 | 0.35 | 0.36 | 0.35 | 0.35 | 0.35 | 0.34 |

1. A concentrated aqueous solution comprising a sodium salt of 5,10-methylene-(6R)-tetrahydrofolic acid and an alkali metal sulfate, which concentrated aqueous solution does not contain citrate or citric acid or any further chemotherapeutic agents.

2. The concentrated aqueous solution according to claim 1, which consists of the sodium salt of 5,10-methylene-(6R)-tetrahydrofolic acid, sodium sulfate, water and optional osmolality correcting additives.

3. The concentrated aqueous solution according to claim 1 wherein a molar ratio of alkali metal sulfate:sodium salt of 5,10-methylene-(6R)-tetrahydrofolic acid is from 0.4:1.0 to 1:2.

4. The concentrated aqueous solution according to claim 1, which contains more than 60 mg sodium salt of 5,10-methylene-(6R)-tetrahydrofolic acid per ml.

5. The concentrated aqueous solution according to claim 1, which contains the sodium salt of 5,10-methylene-(6R)-tetrahydrofolic acid of a purity greater than 98%.

6. A reconstituted product obtained by diluting the concentrated aqueous solution of claim 1 in water or a liquid pharmaceutically acceptable vehicle.

7. The reconstituted product according to claim 6, wherein the water is sterile water for injection.

8. The reconstituted product according to claim 6, further comprising a pharmaceutically acceptable carrier.

9. The reconstituted product according to claim 6, further comprising an additional pharmaceutically acceptable active ingredient.

10. The reconstituted product according to claim 6, further comprising a buffer, and/or one or more osmolality correcting excipients, or both.

11. The reconstituted product according to claim 6 for use in the treatment of cancer or in cancer therapy.

12. The concentrated aqueous solution according to claim 1, which contains more than 65 mg sodium salt of 5,10-methylene-(6R)-tetrahydrofolic acid per ml.

13. The concentrated aqueous solution according to claim 1, which contains more than 70 mg sodium salt of 5,10-methylene-(6R)-tetrahydrofolic acid per ml.

14. The concentrated aqueous solution according to claim 1, which contains more than 75 mg sodium salt of 5,10-methylene-(6R)-tetrahydrofolic acid per ml.

15. The concentrated aqueous solution according to claim 1, which contains at least 80 mg sodium salt of 5,10-methylene-(6R)-tetrahydrofolic acid per ml.

16. The concentrated aqueous solution according to claim 1, wherein the chemotherapeutic agent is 5-fluorouracil (5-FU) or a derivative.

17. The concentrated aqueous solution according to claim 1, wherein the chemotherapeutic agent is an antifolate.
